(19) United States
(12) Patent Application Publication
(10) Pub. No.: US 2003/0068658 A1
(43) Pub. Date: Apr. 10, 2003

(54) Measurements of marker of oxidative stress in plasma immunoblotting

(75) Inventors: Ali Keshavarzian, Chicago, IL (US); Ali Banan, Chicago, IL (US)

Correspondence Address: WELSH & KATZ, LTD, 120 S RIVERSIDE PLAZA, 22ND FLOOR, CHICAGO, IL, 60606, US

(73) Assignee: Rush Presbyterian St. Luke's Medical Center, Chicago, IL

(21) Appl. No.: 10/263,207

(22) Filed: Oct. 2, 2002

Related U.S. Application Data
(60) Provisional application No. 60/326,559, filed on Oct. 2, 2001.

(51) Int. Cl. G01N 33/53; G01N 33/542; G01N 33/537; G01N 33/543

(52) U.S. Cl. 435/7.9; 435/7.93

(57) ABSTRACT

The presence of oxidative stress in a patient is determined by immobilizing plasma proteins onto a support, derivatizing any oxidized proteins with 2,4-dinitrophenylhydrazine (DNPH), contacting the derivatized plasma proteins with anti-DNPH antibody, then measuring the amount of immunocomplex formed. In a preferred embodiment, the plasma proteins are bound to a membrane, derivatized with DNPH, contacted with anti-DNPH antibody, with the amount of immunocomplex formed being determined by contacting the immunocomplex with a second antibody labeled with horseradish peroxidase in conjunction with chemiluminescence.

### TECHNICAL FIELD

This invention relates to methods for assaying oxidative stress in a patient. More particularly, this invention relates to immunological methods for detecting oxidized proteins in plasma.

### BACKGROUND OF THE INVENTION

Oxidative damage to proteins is associated with several diseases in humans including Alzheimer's disease [C. Smith et al., Proc. Natl. Acad. Sci. USA 88, 10540-10543 (1991)], rheumatoid arthritis [M. L. Chapman et al., J. Rheumatol. 16, 15-18 (1989)], and inflammatory bowel disease [L. Lih-Brody et al., Dig. Dis. Sci. 41, 2078-2086 (1996)]. Protein oxidation can result in the formation of carbonyl groups (aldehydes and ketones) in certain amino acids. The amount of carbonyl groups on the proteins can be correlated with the level of oxidative stress in the patient.

The classic approach for the detection of protein carbonyl groups involves the reaction of the protein's carbonyl group with 2,4-dinitrophenyl-hydrazine (DNPH) followed by spectrophotometric quantification of the resulting acid hydrazones at 370 nm [R. L. Levine et al., Methods Enzymol. 233, 346-357 (1994)]. This method has the disadvantages in that large amounts of pure protein are required for detection and that significant interference or high background can be present due to the presence of DNA and unreacted DNPH. Another method for carbonyl analysis is HPLC separation followed by spectroscopy at 357 nm; however this technique can be problematic for crude protein mixtures where resolution of low-and medium-molecular weight proteins is difficult. Carbonyl groups can also be detected by labeling with tritiated borohydride [R. L. Levine et al., Methods Enzymol. 186, 464-478 (1990)], but this method suffers from high background, poor specificity, and problems with crude protein samples.

Immunochemical techniques have been previously applied to the detection of carbonyl groups in proteins that have been purified and separated by polyacrylamide gel electrophoresis [E. Schacter et al., Free Radical Biol. Med. 17, 429-437 (1994); and C. E. Robinson et al., Analyt. Biochem. 266, 48-57 (1999)]. This method suffers in that the proteins are denatured and analysis of crude protein samples is difficult and laborious.

The present invention addresses and provides one solution to these problems by simple yet sensitive methods that assay for oxidative stress in whole plasma using immunological detection techniques for measuring the amount of oxidized proteins.

### SUMMARY OF THE INVENTION

The presence of oxidative stress in a patient can be determined by a method employing an immunological assay for oxidized proteins in the plasma.

A contemplated method for assaying for the presence of oxidative stress in a patient comprising the following steps. (i) Protein from a predetermined amount of plasma from a patient is bound to a support, forming a support-bound plasma protein. (ii) The support-bound plasma protein is reacted with 2,4-dinitrophenylhydrazine (DNPH) to form a derivatized support-bound plasma protein. (iii) The derivatized support-bound plasma protein is contacted with anti-DNPH antibody and the contact is maintained for a time period sufficient to form an immunocomplex between the derivatized support-bound plasma protein and the anti-DNPH antibody. (iv) The amount of immunocomplex present is determined and compared to the amount of immunocomplex present in the same quantity of a standard serum sample. An amount of immunocomplex present greater than that present in the standard sample in excess of experimental error indicates the presence of oxidative stress in the patient.

The present invention provides advantages in being simpler and easier to use than prior methods because here whole plasma from a patient is utilized instead of tissue or cell homogenates.

The present invention also overcomes inherent problems of the prior art including the inability to analyze crude protein samples and the need to denature the proteins before immunological detection. Still further benefits and advantages of the invention will be apparent to those skilled in this art from the detailed description that follows.

### DETAILED DESCRIPTION OF THE INVENTION

The present invention is directed to a method of detecting oxidative stress in a patient. One contemplated method assays for the presence of oxidative stress in a patient comprising the following steps. (i) Protein from a predetermined amount of plasma from a patient is bound to a support, forming a support-bound plasma protein. (ii) The support-bound plasma protein is reacted with 2,4-dinitrophenylhydrazine (DNPH) to form a derivatized support-bound plasma protein. (iii) The derivatized support-bound plasma protein is contacted with anti-DNPH antibody and the contact is maintained for a time period sufficient to form an immunocomplex between the derivatized support-bound plasma protein and the anti-DNPH antibody. (iv) The amount of immunocomplex present is determined and compared to the amount of immunocomplex present in the same quantity of a standard serum sample. An amount of immunocomplex present greater than that present in the standard sample in excess of experimental error indicates the presence of oxidative stress in the patient.

In a preferred embodiment, the support is a membrane such as a nitrocellulose membrane as commonly used in Western blot techniques. In another embodiment, the support is a gel matrix, such as a polyacrylamide gel for example. In yet another embodiment, the support is a porous particle similar to those found in column chromatography including HPLC such as sephadex, sepharose, and silica. In a different embodiment, the support is a plastic surface of a microtiter plate such as polystyrene or polycarbonate used in ELISA techniques. In yet another method, the support is a biological molecule such as a nucleic acid (DNA or RNA), proteins or peptides, or antibodies or antigens.

The amount of immunocomplex formed by the reaction of the plasma protein with DNPH is preferably determined by ultra-violet spectroscopy using the absorbance at 370 nm. In a different embodiment, the amount of immunocomplex present is determined by radiography by use of radiolabeled anti-DNPH antibodies. In yet another embodiment, the amount of immunocomplex present is determined by fluorescence spectroscopy.

In a more preferred embodiment, the amount of immunocomplex present is determined by binding a second antibody to the immunocomplex and measuring the amount of bound secondary antibody. Preferably, the second antibody is labeled with a fluorescent tag. In another embodiment, the second antibody is labeled with a radioactive molecule. In the most preferred embodiment, the second antibody is labeled with horseradish peroxidase. The amount of horseradish peroxidase can then be quantified by chemiluminescence techniques.

Each of the patents and articles cited herein is incorporated by reference. The use of the article “a” or “an” is intended to include one or more.

From the foregoing, it will be observed that numerous modifications and variations can be effected without departing from the true spirit and scope of the present invention. It is to be understood that no limitation with respect to the specific examples presented is intended or should be inferred. The disclosure is intended to cover by the appended claims modifications as fall within the scope of the claims.

We claim:

1. A method assaying for the presence of oxidative stress in a patient comprising the steps of:
(i) binding protein from a predetermined amount of plasma from a patient to a support forming a support-bound plasma protein;
(ii) reacting the support-bound plasma protein with 2,4-dinitrophenylhydrazine (DNPH) to form a derivatized support-bound plasma protein;
(iii) contacting the derivatized support-bound plasma protein with anti-DNPH antibody and maintaining that contact for a time period sufficient to form an immunocomplex between the derivatized support-bound plasma protein and the anti-DNPH antibody; and
(iv) determining the amount of immunocomplex present and comparing that amount to the amount of immunocomplex present in the same quantity of a standard serum sample, the amount greater than that present in the standard serum sample in excess of experimental error indicating the presence of oxidative stress in the patient.

2. The method of claim 1 wherein the support is a membrane.

3. The method of claim 1 wherein the support is a gel matrix.

4. The method of claim 1 wherein the support is a porous particle.

5. The method of claim 1 wherein the support is a plate.

6. The method of claim 1 wherein the support is a biological molecule.

7. The method of claim 1 wherein the support is a nucleic acid.

8. The method of claim 1 wherein the support is a protein.

9. The method of claim 1 wherein the support is an antibody.

10. The method of claim 1 wherein the support is an antigen.

11. The method of claim 1 wherein the amount of immunocomplex present is determined by ultra-violet spectroscopy.

12. The method of claim 1 wherein the amount of immunocomplex present is determined by radiography.

13. The method of claim 1 wherein the amount of immunocomplex present is determined by fluorescence spectroscopy.

14. The method of claim 1 wherein the amount of immunocomplex present is determined by binding the immunocomplex to a second antibody and measuring the amount of bound secondary antibody.

15. The method of claim 14 wherein the second antibody is labeled with a fluorescent tag.

16. The method of claim 14 wherein the second antibody is labeled with a radioactive molecule.

17. The method if claim 14 wherein the second antibody is labeled with horseradish peroxidase.

18. A method assaying for the presence of oxidative stress in a patient comprising the steps of:
(i) binding protein from a predetermined amount of plasma from a patient to a membrane to form a membrane-bound plasma protein;
(ii) reacting the membrane-bound plasma protein with 2,4, dinitrophenylhydrazine (DNPH) to form a derivatized membrane-bound plasma protein;
(iii) contacting the derivatized membrane-bound plasma protein with anti-DNPH antibody and maintaining that contact for a time period sufficient to form an immunocomplex between the derivatized membrane-bound plasma protein and the anti-DNPH antibody; and
(iv) determining the amount of immunocomplex formed wherein the amount formed is determined by binding the immunocomplex to a second antibody that is labeled with horseradish peroxidase and measuring the amount of bound secondary antibody labeled with horseradish peroxidase and comparing that amount to the amount of bound secondary antibody labeled with horseradish peroxidase present in the same quantity of a standard serum sample, the amount greater than that present in the standard serum sample in excess of experimental error indicating the presence of oxidative stress in the patient.
